(19) United States
(12) Patent Application Publication
(10) Pub. No.: US 2018/0305418 A1
(43) Pub. Date: Oct. 25, 2018

(54) THERMOCONDUCTIVE MATERIAL, HEAT SINK, HEAT SPREADER, METHOD FOR PRODUCING HEAT SPREADER, AND METHOD FOR PRODUCING THERMOCONDUCTIVE MATERIAL

(71) Applicants: SEKISUI CHEMICAL CO., LTD., Osaka (JP); Rowan University, Glassboro, NJ (US)

(72) Inventors: Tomoaki KATAGIRI, Tsukuba-shi (JP); Rasika DASANAYAKE ALUTHGE, Tsukuba-shi (JP); Xiao HU, Glassboro, NJ (US); Samuel LOFLAND, Springfield, PA (US)

(73) Assignees: SEKISUI CHEMICAL CO., LTD., Osaka (JP); Rowan University, Glassboro, NJ (US)

(21) Appl. No.: 15/769,229

(22) PCT Filed: Oct. 28, 2016
(86) PCT No.: PCT/JP2016/082895

§ 371 Date: Apr. 18, 2018

Related U.S. Application Data
(60) Provisional application No. 62/248,311, filed on Oct. 30, 2015.

(51) Int. Cl. C07K 14/425; C08J 5/00; G01N 23/207; B29C 43/00

(52) U.S. Cl. CPC C07K 14/425; B29C 43/006; G01N 23/2076; C08J 5/005

(57) ABSTRACT

Disclosed is a thermoconductive material comprising a protein and having a thermal conductivity of more than 0.6 W/(m·K). Also disclosed is a method for producing a thermoconductive material comprising a protein at least a part of which forms crystalline β-pleated sheets, the crystalline β-pleated sheets being present in an amount of 10% by weight or more, based on the total weight of the protein, and the thermoconductive material having a thermal conductivity of more than 0.6 W/(m·K), the method comprising dissolving a raw material protein in a formic acid solution containing calcium chloride to obtain a protein solution, and evaporating the formic acid from the protein solution.

### FIELD OF THE INVENTION

The present invention relates to a thermoconductive material including crystalline β-sheets of a protein, a heat sink and a heat spreader each using the thermoconductive material, a method for producing a heat spreader, and a method for producing a thermoconductive material.

### DESCRIPTION OF RELATED ART

The thermal conductivity (thermal conductance) is a property of materials to conduct heat. There is a huge demand in industry for materials having excellent thermal conductivity; however, known thermoconductive materials have one or more defects. As examples of known thermoconductive materials, there can be mentioned metals, oxides thereof and carbonaceous materials. Metals and oxides thereof are generally defective in that they are hard and brittle, and such physical properties cannot be easily controlled. Further, carbonaceous materials and metals have high electrical conductivity as well, and hence are not appropriate for some applications. Some synthetic resin materials are known to have high thermal conductivity, which however have a problem in that such materials are flammable or leave toxic residue. It is also known to incorporate inorganic fillers into the synthetic resin materials in order to increase the thermal conductivity of the synthetic resin materials; however, this technique has a problem in that the resin materials become hard and brittle as the amount of inorganic fillers is increased (see Non-patent document 1).

Biological materials derived from natural, living organisms have been drawing attention as renewable materials with less environmental load, and are important materials due to excellent properties thereof such as high tensile strength, extensibility, toughness, chemical stability, lightness in weight, electrical conductivity and biocompatibility. Biological materials in general have glass transition temperatures (T_{g}) higher than those of synthesis resins. For example, a silk derived from Bombyx mori has a T_{g} of about 178° C. This T_{g} is much higher than those of many synthetic resins (see Non-patent document 2). However, it cannot be said that the thermal conductivity of biological materials known to date is high enough.

Further, Patent Document 1 discloses a method for producing a thermoconductive material, which includes heating a silk protein by pulse electric current sintering, followed by press treatment. In this method, first, an aqueous solvent (water, glycerol, etc.) or a water-soluble polymer (polyvinyl pyrrolidone, polyvinyl alcohol, poly hydroxy methacrylate, etc.) is added to a silk protein, and the resulting is subjected to heat treatment and press treatment. This patent document states that a high thermal conductivity comparable to the high-density polyethylene resin (0.38 to 0.60 W/(m−K)) is achievable; however, by the technique of this patent document, a thermal conductivity exceeding this level is not available (in the Example, the thermal conductivity is 0.44 W/(m−K)).

In this situation, it is strongly demanded to develop a biological material which not only has high thermal conductivity and structural flexibility, but also imposes less environmental load.

### DOCUMENTS OF RELATED ART

### Patent Documents

[Patent Document 1] Japanese Unexamined Patent Application Publication No. 2007-277481

### Non-Patent Documents

[Non-patent Document 1] “Outlook of Thermal Interface Material (TIM) Market 2015”, Japan Marketing Survey Co., Ltd.
[Non-patent Document 2] “Protein-based composite materials” Hu, et al. Materials Today, 2012, 15 (5), 208-215

### SUMMARY OF THE INVENTION

The present invention has been made in view of the aforementioned situation, and provides a thermoconductive material having high thermal conductivity and structural flexibility, a heat sink or a heat spreader including the thermoconductive material, a method for producing such a heat spreader, and a method for producing the thermoconductive material.

Specifically, the present invention relates to the following.

[1] A thermoconductive material comprising a protein and having a thermal conductivity of more than 0.6 W/(m·K).
[2] The thermoconductive material, wherein at least a part of the protein forms crystalline β-pleated sheets, the crystalline β-pleated sheets being present in an amount of 10% by weight or more, based on the total weight of the protein.
[3] The thermoconductive material according to [1], which is obtained by a treatment for increasing the amount of the β-pleated sheets.
[4] The thermoconductive material according to [3], wherein the treatment is a press treatment.
[5] The thermoconductive material according to any one of [1] to [4], wherein the protein is fibroin or corn zein.
[6] The thermoconductive material according to any one of [1] to [5], which has a shape of a fiber, a film, a block, a column, a spheroid or a sphere.
[7] The thermoconductive material according to any one of [1] to [6], which has a shape of particles, granules or pellets.
[8] A heat sink comprising the thermoconductive material of any one of [1] to [7].
[9] A heat spreader comprising the thermoconductive material of any one of [1] to [7].
[10] A method for producing a heat spreader, comprising pressing the thermoconductive material of any one of [1] to [7] against an installation surface of a base to obtain a heat spreader comprising the thermoconductive material installed on the installation surface of the base.
[11] A method for producing a thermoconductive material including a protein at least a part of which forms crystalline β-pleated sheets,

the crystalline β-pleated sheets being present in an amount of 10% by weight or more, based on the total weight of the protein, and

the thermoconductive material having a thermal conductivity of more than 0.6 W/(m·K),

the method comprising dissolving a raw material protein in a formic acid solution containing calcium chloride to obtain a protein solution, and evaporating the formic acid from the protein solution.

[12] The method according to [11], which further comprises immersing the thermoconductive material in a polar solvent to cause at least a part of the calcium chloride or the formic acid to elute from the thermoconductive material into the polar solvent.
[13] The method according to [12], which further comprises pressing the thermoconductive material after having caused at least a part of the calcium chloride or the formic acid to elute from the thermoconductive material into the water.

The thermoconductive material of the present invention has high thermal conductivity, so that the thermoconductive material can absorb heat from a base on which the thermoconductive material is installed, and then conduct heat efficiently to air or other materials. Further, the thermoconductive material of the present invention is structurally flexible and, hence, exhibits high adhesion to a base when being pressed against the base on which the thermoconductive material is installed. As a result, excellent thermal conduction is achieved between the thermoconductive material and the base. Therefore, the thermoconductive material is suited for use as a heat sink or a heat spreader.

According to the production method of the present invention, a thermoconductive material having excellent thermal conductivity and structural flexibility can be easily produced.

### BRIEF DESCRIPTION OF THE DRAWINGS

FIG. 1 shows a procedure for producing a thermoconductive material in the form of a thin film by using Tussah silk as an example of a raw material protein.

FIG. 2 shows SEM images of surfaces and cross-sections of film-shaped thermoconductive materials formed of respective proteins, which are produced by cast method in the Example.

FIG. 3 shows schematic views of molecular chains constituting a nanostructure present in a film-shaped thermoconductive film according to one embodiment of the present invention.

FIG. 4A shows FTIR absorption spectra of film-shaped thermoconductive materials which are respectively formed of various raw material proteins (i.e., Tussah silk, Bombyx mori silk, Eri silk, Muga silk, Thai silk and corn zein), the spectra being obtained after the thermoconductive materials are press-treated.

FIG. 4B shows an enlarged view of the FTIR spectra in a region between 1600 and 1700 cm⁻¹, which corresponds to the amide vibration.

FIG. 5 shows a procedure for preparation of measurement of the thermal conductivity of a film-shaped thermoconductive material according to one embodiment of the present invention.

FIG. 6 is a schematic view showing the thermoconductive material which is being press-treated for use (installation).

FIG. 7 is a graph showing the results of thermal conductivity measurement of film-shaped thermoconductive materials (using Tussah silk) in the Examples.

FIG. 8 is a graph showing the results of thermal conductivity measurement of film-shaped thermoconductive materials (using Mori silk) in the Examples.

FIG. 9 is a graph showing the results of thermal conductivity measurement of film-shaped thermoconductive materials (using Eri silk) in the Examples.

FIG. 10 is a graph showing the results of thermal conductivity measurement of film-shaped thermoconductive materials (using Muga silk) in the Examples.

FIG. 11 is a graph showing the results of thermal conductivity measurement of film-shaped thermoconductive materials (using Thai silk) in the Examples.

FIG. 12 is a graph showing the results of thermal conductivity measurement of film-shaped thermoconductive materials (using corn zein) in the Examples.

### DETAILED DESCRIPTION OF THE INVENTION

Herein below, the present invention is described with reference to preferred embodiments thereof which, however, should not be construed as limiting the scope of the present invention.

[symbol]

Thermoconductive Material

[symbol]

In the first aspect of the present invention, there is provided a thermoconductive material including a protein.

The thermoconductive material of the first embodiment of the present invention is a solid comprising a protein and having a thermal conductivity of more than 0.6 W/(m·K). It is preferred that at least a part of the protein forms crystalline β-pleated sheets. The amount of the crystalline β-pleated sheets is preferably 10% by weight or more, based on the total weight of the protein constituting the thermoconductive material. When the amount of crystalline β-pleated sheets is 10% by weight or more, the thermal conductivity of more than 0.6 W/(m·K) (Watts/meter per Kelvin) can be easily achieved.

The amount of protein contained in the thermoconductive material of the present embodiment is not particularly limited, but is preferably 10 to 100% by weight, more preferably 50 to 100% by weight, and still more preferably 80 to 100% by weight, based on the total weight of the thermoconductive material.

The determination of the amount of protein contained in the thermoconductive material of the present embodiment can be performed by conventional methods. Specific examples of the determination method include spectrophotometric methods such as the BCA method, Bradford method, Lowry method and Biuret method. Alternatively, the amount can also be determined based on the bands obtained in gel electrophoresis of a sample.

In the present invention, the amount of crystalline β-pleated sheets contained in the thermoconductive material of the present embodiment is a value measured by the Fourier transform infrared spectroscopy (FTIR).

Here, the β-pleated sheets are indicated as being “crystalline” because the β-pleated sheets contained in the thermoconductive material of the present invention are found to be crystalline based on the X-ray diffraction (XRD) pattern thereof. The reason why the XRD pattern indicates that the β-pleated sheets are crystalline is generally considered to be that many β-pleated sheets are densely packed together so as to form a periodic structure where the surfaces of many pleats of the β-pleated sheets diffract X-ray. However, it is generally difficult to accurately measure the amount of crystalline β-pleated sheets contained in the thermoconductive material by the XRD analysis. Nevertheless, the FTIR analysis enables an easy and accurate measurement of the amount of crystalline β-pleated sheets contained in the thermoconductive material. With respect to the specific method for measurement by the FTIR analysis, reference can be made to, for example, “Determining Beta-Sheet Crystallinity in Fibrous Proteins by Thermal Analysis and Infrared Spectroscopy” Xiao Hu, David Kaplan, and Peggy Cebe; Macromolecules 2006, 39, 6161-6170. In the FTIR analysis, a peak is observed within the wavelength range of 1620 to 1640 cm⁻¹, which peak has an intensity corresponding to the amount of crystalline β-pleated sheets. Further, in the FTIR analysis, a peak is observed within the wavelength range of 1640 to 1660 cm⁻¹, which has an intensity corresponding to the amount of amorphous β-pleated sheets. Thus, the FTIR analysis can distinguish between the crystalline β-pleated sheets and the amorphous β-pleated sheets, and determine the respective amounts thereof.

With respect to the thermoconductive material of the present embodiment, the thermal conductivity thereof tends to increase as the amount of crystalline β-pleated sheets increases. For this reason, the amount of crystalline β-pleated sheets is preferably 10% by weight or more, more preferably 20% by weight or more, and more preferably 30% by weight or more, still more preferably 40% by weight or more, and most preferably 50% by weight or more, based on the total weight of the protein contained in the thermoconductive material. By increasing the amount of crystalline β-pleated sheets, the thermal conductivity of the thermoconductive material of the present embodiment can be improved irrespective of the direction of the measurement.

The upper limit of the amount of crystalline β-pleated sheets is not particularly limited, and can theoretically be 100% by weight when the whole of the protein constituting the thermoconductive material of the present embodiment is in the form of crystalline β-pleated sheets. However, secondary structures that a protein generally form include an α-helix, an amorphous β-pleated sheet, a turn and a random coil in addition to the crystalline β-pleated sheet; therefore, in many cases, the amount of crystalline β-pleated sheets is less than 100% by weight, and is more likely to be less than 90% by weight. Incidentally, the influence of the secondary structures other than the crystalline β-pleated sheet on the thermal conductivity of the thermoconductive material of the present embodiment is not necessarily clear.

The β-pleated sheets in the crystalline β-pleated sheets may possess either a parallel β-sheet structure or an anti-parallel β-sheet structure.

The amount of crystalline β-pleated sheets measured by the FTIR analysis correlates with the crystallinity evaluated in terms of degree of the XRD peak intensity. Specifically, the thermoconductive material having a higher crystallinity tends to have a larger amount of crystalline β-pleated sheets.

The thermoconductive material of the present embodiment may further include at least one optional component selected from the group consisting of organic materials other than proteins, inorganic materials, and other materials.

Examples of the organic materials include thermosetting or thermoplastic synthetic resins. A thermoconductive composite material obtained by blending the thermoconductive material of the present embodiment with a synthetic resin can be designed to exhibit excellent properties such as fabricability, rigidity and elasticity. The aforementioned synthetic resins may be synthetic resins formed of petroleum-based materials, biodegradable synthetic resins or the like.

Specific examples of the synthetic resins include synthetic resins containing no halogens, such as polyesters, vinyl polymers and polyolefins; and halogen-containing synthetic resins such as polyvinyl chloride and polytetrafluoroethylene. Further, an electroconductive polymer may be blended into the thermoconductive material of the present embodiment for enhancing the thermal conductivity of the thermoconductive material.

Specific examples of the inorganic materials include metals such as aluminum, copper, iron and gold; metal oxides such as a titanium oxide, a zinc oxide, a magnesium oxide and a silicon oxide; glass; and ceramics.

Examples of the aforementioned other materials include ionic compounds, low molecular weight compounds, polymers and biological compounds, which are other than the aforementioned organic and inorganic materials.

The thermal conductivity of the thermoconductive material of the present embodiment is preferred to be as high as possible from the viewpoint of use thereof as a heat sink or a heat spreader.

The thermoconductive material of the present embodiment can exhibit high thermal conductivity even when the thermoconductive material contains substantially no other material than protein.

When the amount of protein is 99% by weight or more, based on the total weight of the thermoconductive material of the present embodiment, the thermal conductivity of the thermoconductive material is, for example, preferably more than 0.6 W/(m·K), more preferably 1.0 W/(m·K) or more, still more preferably 2.0 W/(m·K) or more, still more preferably 3.0 W/(m·K) or more, still more preferably 4.0 W/(m·K) or more, especially preferably 5.0 W/(m·K) or more, and most preferably 6.0 W/(m·K) or more. The upper limit of the thermal conductivity of the thermoconductive material of the present embodiment is not particularly limited.

The thermoconductive material of the present embodiment may exhibit poor solubility in water after being solidified. Therefore, the surface of the solidified thermoconductive material can be washed with water. The poor solubility in water exhibited by the thermoconductive material is advantageous in that, when the thermoconductive material is used while being in contact with water, the shape of the thermoconductive material can be retained.

The raw material protein used for forming the thermoconductive material of the present embodiment may be obtained by purifying the extracts from natural living organisms or mutant living organisms genetically engineered by conventional methods, or may be obtained by chemical synthesis. With respect to the raw material protein, the molecular weight, amino acid sequence (primary structure), three-dimensional structure (tertiary structure) and subunit structure (quaternary structure) are not particularly limited. It is preferred that the molecules of the raw material protein form β-pleated sheets (secondary structure) or crystalline β-pleated sheets, each of which may be formed by a single protein molecule or a plurality of different protein molecules. The crystalline β-pleated sheets can be detected and determined by the aforementioned FTIR analysis.

The amount of crystalline β-pleated sheets may be lower in the raw material protein than in the thermoconductive material of the present embodiment. The reason therefor is that the amount of crystalline β-pleated sheets in the raw material protein can be increased by the below-mentioned method for producing the thermoconductive material.

As the raw material protein, it is preferred to use a protein with its quaternary structure being fibrous, and it is more preferred to use a fibrous protein detectable by the XRD analysis. Specific examples of such proteins include collagen, keratin, elastin, fibroin, and a protein constituting a spider silk. Further, corn zein can also be mentioned as a preferred example of the raw material protein.

The shape and size of the thermoconductive material of the present embodiment are not particularly limited, and the shape and size as exemplified below in the explanation of the production method can be mentioned as preferred examples thereof.

[symbol]

Method for Producing Thermoconductive Material

[symbol]

In the second aspect of the present invention, there is provided a method for producing a thermoconductive material.

The method of the first embodiment of the present invention is a method for producing a thermoconductive material including crystalline β-pleated sheets of a protein in an amount of 10% by weight or more, based on the total weight of the protein, the method including dissolving a raw material protein in a formic acid (HCOOH) solution containing calcium chloride (CaCl₂) to obtain a protein solution, and evaporating the formic acid from the protein solution to solidify the raw material protein.

This method enables a production of a thermoconductive material having a thermal conductivity of more than 0.6 W/(m·K).

The formic acid solution (reagent grade, ≥98%) used for dissolving the raw material protein contains calcium chloride. The concentration of calcium chloride is not particularly limited but, for example, is preferably 0.1 to 15% by weight, more preferably 1.0 to 10% by weight, still more preferably 2.0 to 8.0% by weight, and most preferably 2.0 to 6.0% by weight, based on the total weight of the formic acid solution.

When the concentration of calcium chloride is 0.1% by weight or more, not only can the solubility of the raw material protein be increased, but the amount of crystalline β-pleated sheets in the thermoconductive material after removal of the formic acid can be easily increased. When the concentration of calcium chloride is 15% by weight or less, the amount of residual calcium chloride in the thermoconductive material can be easily decreased by removal of calcium chloride from the thermoconductive material.

The concentration of the raw material protein in the protein solution is not particularly limited but, for example, is preferably 1 to 50% by weight, more preferably 3 to 40% by weight, still more preferably 5 to 20% by weight, and most preferably 15 to 30% by weight, based on the total weight of the protein solution. These upper limits and lower limits can be arbitrarily combined in any manner depending on the use of the thermoconductive material of the present invention, etc.

When the concentration of the raw material protein is not less than the lower limit of the aforementioned range, the protein solution exhibits appropriate viscosity so that the thermoconductive material can be easily formed into a desired shape.

When the concentration of the raw material protein is not more than the upper limit of the aforementioned range, the raw material protein can be completely dissolved and the amount of crystalline β-pleated sheets in the thermoconductive material after removal of the formic acid can be easily increased.

Incidentally, by the use of an aqueous solvent (water, glycerol, etc.) or a water-soluble polymer (polyvinyl pyrrolidone, polyvinyl alcohol, poly hydroxy methacrylate, etc.) as used in the aforementioned Patent Document 1, the amount of crystalline β-pleated sheets in the thermoconductive material cannot be controlled. This is considered to be the reason why a sufficiently high thermal conductivity cannot be achieved by the technique of Patent Document 1.

The method for evaporating the formic acid contained in the protein solution from the thermoconductive material is not particularly limited, and examples thereof include a method comprising casting the protein solution onto a substrate, and evaporating the formic acid, thereby forming the thermoconductive material in the form of a film (sheet) on the substrate. Further, the protein solution may be cast into a mold having a predetermined shape, followed by evaporation of the formic acid in the mold, thereby obtaining the thermoconductive material having a shape corresponding to the mold. Alternatively, by spray drying the protein solution, the thermoconductive material in the form of particles or granules can be obtained.

The type of the aforementioned substrate is not particularly limited, and examples thereof include substrates made of resins such as polydimethylsiloxane (PDMS) and polytetrafluoroethylene (PTFE) (e.g., Teflon), and substrates made of metals such as aluminum and copper.

When the thickness (depth) of the protein solution cast on the substrate is increased, the film thickness of the thermoconductive material can be increased. Specifically, for example, when a protein solution with the raw material protein concentration of 10% by weight is cast to a thickness of 1.0-1.2 mm on the substrate, and then left to dry, the thermoconductive film in the form of a film having a thickness of 0.4-0.6 mm can be obtained. In this instance, the drying of the protein solution cast on the substrate may be accelerated by heating the substrate, blowing hot air against the substrate, placing the substrate in vacuo, etc.

The shape of the mold is not particularly limited. For example, the mold may have a shape such that the thermoconductive material can be shaped into a fiber, a film, a block, a column, a spheroid or a sphere. After the protein solution is cast into the mold, the drying of the solution may be accelerated by heating the mold, evacuating gas from the mold to vacuum dry the content thereof, etc. Further, by compressing the protein solution by the mold, the thermoconductive material having a desired shape corresponding to the mold can be easily obtained. By compressing the thermoconductive material (i.e., press treatment), the amount of crystalline β-pleated sheets in the thermoconductive material can be increased so as to improve the thermal conductivity thereof. When the amount of crystalline β-pleated sheets is increased by an appropriate treatment as described herein, the resulting thermoconductive material exhibits high thermal conductivity irrespective of the direction of the measurement.

By spray drying the protein solution, the formic acid can be removed from the solution so as to obtain the thermoconductive material in the form of particles or granules.

Alternatively, the thermoconductive material in the form of particles, granules or pellets can also be obtained by subjecting the thermoconductive material of any shape such as a fiber, a film, a block, a column, a spheroid or a sphere to a conventional size reduction process such as pulverization, crushing or cutting.

With respect to the thermoconductive material in the form of particles, the average particle diameter thereof is not particularly limited and may be, for example, 1 μm to less than 1 mm.

With respect to the thermoconductive material in the form of granules, the average diameter thereof is not particularly limited and may be, for example, 1 mm to less than 5 mm.

With respect to the thermoconductive material in the form of pellets, the average diameter thereof is not particularly limited and may be, for example, 5 mm to 50 mm.

As a method for measuring the average particle diameter of the thermoconductive material in the form of particles, the following method can be mentioned as an example. With respect to 100 particles observed under an electron microscope, the largest axes of respective particles are measured using a scale bar, and an arithmetic mean value of the largest axes is calculated to obtain the average particle diameter. As a method for measuring the average diameter of the thermoconductive material in the form of granules or pellets, the following method can be mentioned as an example. With respect to 100 granules or pellets (if necessary, observed under an optical microscope or a magnifying glass), the largest axes of respective particles are measured using a scale bar, and an arithmetic mean value of the largest axes is calculated to obtain the average diameter.

With respect to the thermoconductive material obtained by removing the formic acid from the aforementioned protein solution, the thermal conductivity thereof can be remarkably enhanced by subjecting the thermoconductive material to a treatment for increasing the amount of the β-pleated sheets (hereinafter, also referred to as “β sheet increasing treatment”). As regards the reason for this enhancing effect, one factor that can be mentioned is the conversion of amorphous β-pleated sheets into crystalline β-pleated sheets, which occurs due to the increase in the amount of the β-pleated sheets in the thermoconductive material of the present embodiment that is achieved by the aforementioned treatment.

Therefore, there is no particular limitation on the β sheet increasing treatment as far as the amorphous and/or crystalline β-pleated sheets can be increased, and examples thereof include a press treatment in which the thermoconductive material is compressed (pressed) in one direction or in a plurality of arbitrary directions; an extension treatment (stretch treatment); a shear treatment; a bend treatment; a twist treatment. The specific type of the press treatment is not particularly limited, and the examples of the press treatment include a plate press and a roll press.

The method for compressing the thermoconductive material of the present embodiment is not particularly limited, and examples thereof include a method in which the thermoconductive material is placed between upper and lower mold halves, and pressed between the mold halves by any conventional method.

The pressure for the press treatment is not particularly limited but, for retaining the structural flexibility inherently possessed by the thermoconductive material as well as for increasing the amount of crystalline β-pleated sheets and the thermal conductivity, it is preferred that the pressure is such that the thickness of the thermoconductive material is reduced by the press treatment to 90% to 10%, more preferably 50% to 10%, of the thickness before the press treatment.

In the press treatment, it is preferred that the thermoconductive material is held in a state of being pressed for a predetermined period of time. By holding the thermoconductive material in a state of being pressed, the crystalline β-pleated sheets can be increased and also stabilized to reduce the ratio of β-pleated sheets returning to the amorphous state, so that the ratio of β-pleated sheets remaining in the crystalline state even after the pressure is released can be increased.

The time for holding the thermoconductive material in a state of being pressed is not particularly limited. However, on condition that the thermoconductive material is pressed under the aforementioned appropriate pressure, for example, the holding time is preferably at least 1 minute, more preferably at least 10 minutes, and still more preferably at least 30 minutes.

By additionally subjecting the thermoconductive material to a heat treatment and/or a steam treatment (vapor spraying treatment) during the press treatment, the thermal conductivity of the thermoconductive material can sometimes be further enhanced.

For enhancing the effect of improving the thermal conductivity by the β sheet increasing treatment, it is preferred that, as a preliminary treatment, the thermoconductive material is immersed in a polar solvent to cause the polar solvent to permeate into the inside of thermoconductive material, and the resulting polar solvent-containing thermoconductive material is subjected to the β sheet increasing treatment.

The time for keeping the thermoconductive material immersed in the polar solvent is not particularly limited, and is preferred to be appropriately adjusted in view of the shape of the thermoconductive material, etc. For example, when the thermoconductive material is in the form of a film having a thickness within or around the range of 0.1 to 5 mm, it is preferred to keep the thermoconductive material immersed in the polar solvent for 1 minute to 24 hours, more preferably 1 minute to 30 minutes. When the thermoconductive material is thicker than mentioned above, it is preferred that the thermoconductive material is kept immersed in the polar solvent for a longer period of time.

By immersing the thermoconductive material of the present embodiment in the polar solvent, the calcium chloride contained in the thermoconductive material is caused to elute into the polar solvent. Further, when the thermoconductive material also contains residual formic acid, the formic acid is also caused to elute into the polar solvent. In addition to the effective removal of calcium chloride and formic acid, the immersion of the thermoconductive material in the polar solvent has another advantage that the thermal conductivity of the thermoconductive material can be easily increased by the subsequent β sheet increasing treatment of the polar solvent-containing thermoconductive material.

In the case where the aforementioned β sheet increasing treatment is carried out after the thermoconductive material has been immersed in the polar solvent, there is no particular limitation as to the timing of the treatment. For example, when the β sheet increasing treatment is the aforementioned press treatment, the press treatment may be carried out immediately after the immersion or may be carried out after drying the immersed thermoconductive material. For enhancing the effect of increasing the β sheet, it is preferable that the press treatment is carried out before drying the immersed thermoconductive material since the improvement of mobility of the protein molecules by the polar solvent remaining in the thermoconductive material can be expected.

With respect to the polar solvent, there is no particular limitation. For efficiently causing the residual formic acid and/or calcium chloride to elute into the polar solvent, for example, it is preferable to use water, an alcohol type solvent such as methanol, ethanol or glycerol, or the like, and it is more preferable to use water or a mixture thereof with another polar solvent.

With respect to the method for introducing the aforementioned optional components into the thermoconductive material of the present embodiment, there is no particular limitation. For example, the optional components can be introduced into the thermoconductive material by a method in which an appropriate amount of the optional component is added to the protein solution, and the resulting protein solution is solidified by the same method as mentioned above, to thereby obtain the thermoconductive material containing the optional component. As an alternative method, the thermoconductive material is immersed in a liquid containing the optional component to cause the optional component to be adhered to the surface of or permeate into the inside of the thermoconductive material.

The thermoconductive material of the present invention can be widely used as a heat sink or a heat spreader for various apparatuses and electronic appliances that require dissipation of heat. That is, in another aspect of the present invention, there is provided a heat sink or a heat spreader comprising the thermoconductive material of the present invention.

### Examples

### [Production of Film-Shaped Thermoconductive Material]

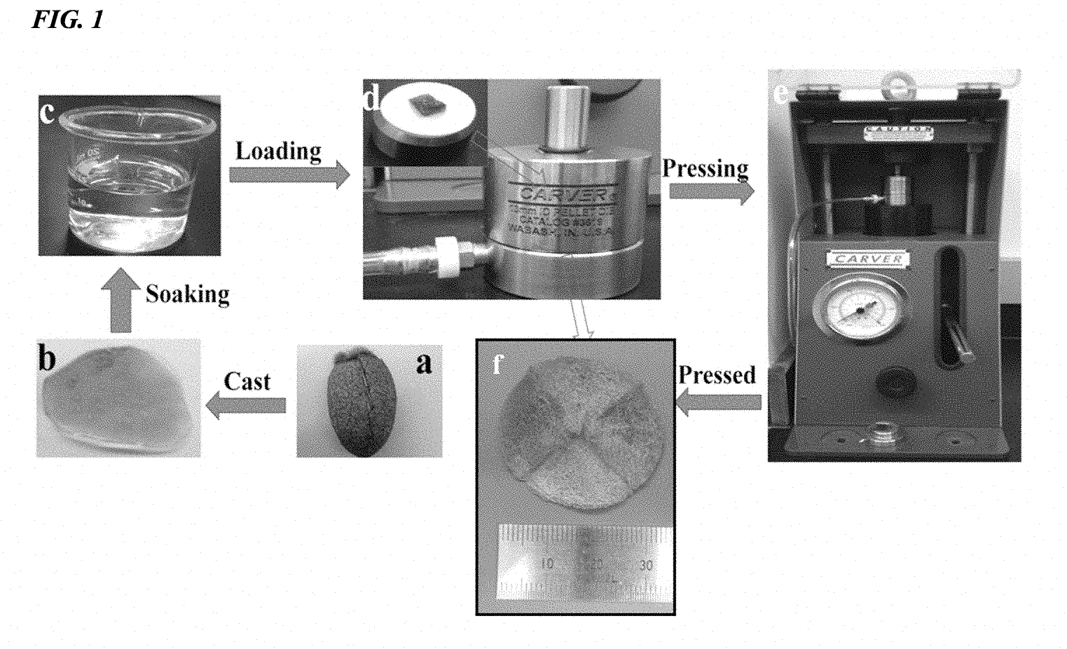

A natural fibrous protein was degummed, purified and washed to obtain a raw material protein. The raw material protein was directly added to a formic acid solution (reagent grade, ≥98%) containing 4% by weight of calcium chloride, followed by shaking for several minutes, to thereby almost completely dissolve the raw material protein. The resulting protein solution was centrifuged at 8,000 rpm for 10 minutes to remove insolubles and gas. Then, the protein solution was cast onto a substrate made of PDMS, followed by drying, to thereby obtain a film-shaped thermoconductive material formed of the raw material protein (“b” in FIG. 1).

The film-shaped thermoconductive material obtained in this Example (hereinafter, simply referred to as “film”) was immersed and kept in water for 5 to 10 minutes to remove residual calcium chloride and formic acid, while also softening the film. The film was almost insoluble in water (“c” in FIG. 1).

The films produced following this procedure were then subjected to press treatment in two different manners as described below.

### (Press Treatment Before Drying)

After removing moisture from the surface of the film, the film was held between plate-shaped upper and lower mold halves (“d” in FIG. 1) attached to a press machine (Model 4350.L, manufactured by Carver, Inc.) and pressed under a pressure of 1.7 ton/cm², where the film was kept in this pressurized state for 30 minutes (“e” in FIG. 1). By thus keeping the film in the pressurized state, it was assumed that the alignment of the molecular chains of the protein constituting the film was stabilized. Finally, the compressed film was allowed to stand at room temperature overnight to completely dry the film, thereby obtaining a film-shape thermoconductive material (“f” in FIG. 1).

(Press Treatment after Drying)

After removing moisture from the surface of the film, the film was allowed to stand at room temperature overnight to completely dry the film. Then, the film was held between plate-shaped upper and lower mold halves (“d” in FIG. 1) attached to a press machine (Model 4350.L, manufactured by Carver, Inc.) and pressed under a pressure of 1.7 ton/cm², where the film was kept in this pressurized state for 30 minutes (“e” in FIG. 1). By thus keeping the film in the pressurized state, it was assumed that the alignment of the molecular chains of the protein constituting the film was stabilized.

FIG. 1 is a schematic view showing a procedure for producing the thermoconductive material in the form of a thin film by using Indian Antheraea mylitta silk (Tussah silk) as an example of the raw material protein.

The production method following this procedure is advantageous not only in that the cost and time for production can be greatly reduced, but also in that the structural flexibility of the film can be improved.

By the method depicted in FIG. 1, films were produced using six types of proteins, that is, Indian Antheraea mylitta silk (Tussah), Philosamia ricini silk (Eri), Antheraea assamensis silk (Muga), Thailand silk (Thai) and Chinese Bombyx mori mulberry silk (Mori), as well as corn zein protein (Zein). Here, the names in the brackets are abbreviations of the respective proteins.

The 6 types of raw material proteins used are all fibrous proteins inherently having nano-filament structures (nanoscale fibrous structures) that can be observed under an electron microscope. The nano-filament structures are maintained and the amount thereof can be increased even after having undergone a processing such as compression, mechanical extension (stretching) or shearing. Therefore, for obtaining the thermoconductive material of the present embodiment which has high crystallinity and well-aligned nano-filament structures so that the thermoconductive material exhibits high thermal conductivity, it is considered to be important that the raw material protein inherently has nano-filament structures. Further, due to the nano-filament structures unique to the fibrous protein used as the raw material, the resulting thermoconductive material can exhibit high thermal conductivity irrespective of the direction of the measurement.

In the production process of the Example, each of the films was produced using the protein solution obtained by dissolving the raw material protein in the formic acid solution containing calcium chloride, whereby the nano-filament structures of the raw material protein can be carried on into the thermoconductive material without being destroyed. The nano-filament structure of the raw material protein is a molecular level platform that imparts the resulting material with excellent thermal conductivity, and this supramolecular structure contributes to the flexibility and fabricability of the thermoconductive material such that the material can be shaped into a flexible film, etc.

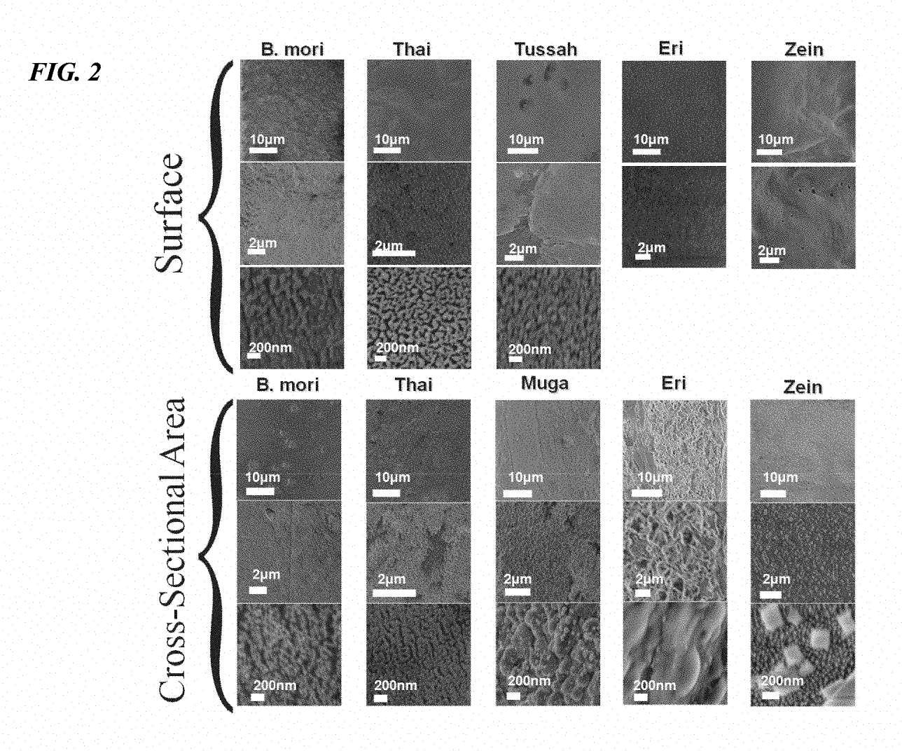

The SEM images of the films prior to the press treatment in the Example are shown in FIG. 2. The SEM images of the respective films have different appearances, which reflect the difference in crystallinity and thermal conductivity. However, the nano-filament structures are commonly observed in the SEM images of the highest magnification (with the scale bar of 200 nm) with respect to all of the films.

In the production method of this Example, the press treatment of the film increased the amount of crystalline β-pleated sheets (crystallinity) in the film and enhanced the thermal conductivity thereof.

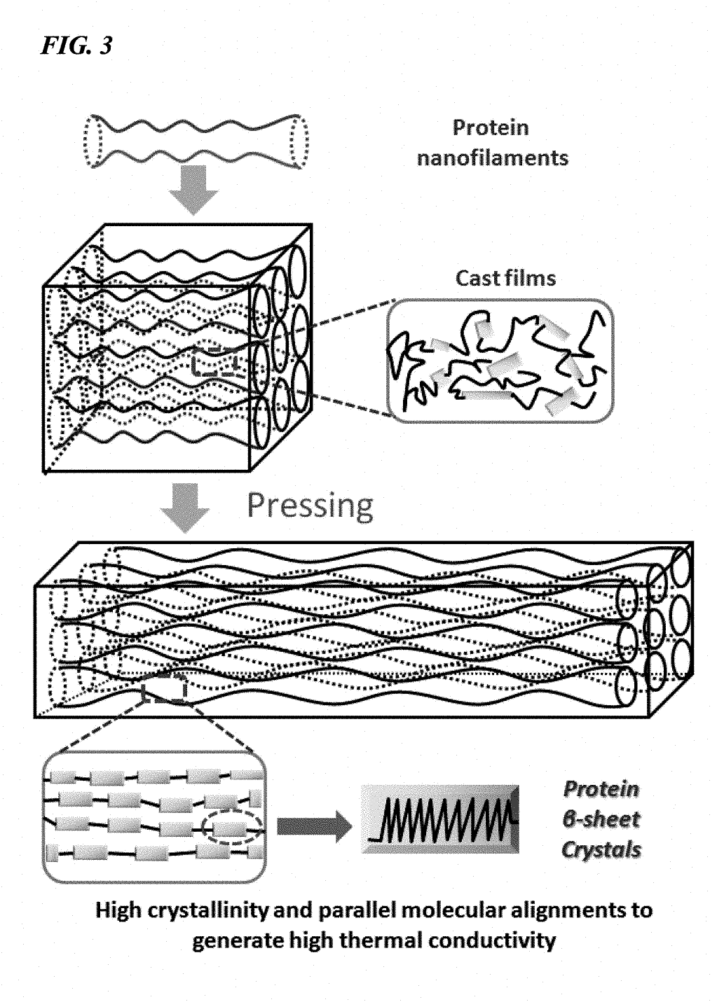

In addition, it was thought that the alignment of the protein molecules and the alignment of the nano-filament structures were modified so as to become uniform (see FIG. 3).

With respect to each of the films produced in this Example, the change in the amount of crystalline β-pleated sheets before and after the press treatment is shown in Tables 1 and 2. Table 1 shows the results with respect to the films press-treated after drying, whereas Table 2 shows the results with respect to the films press-treated before drying. As can be seen from Tables 1 and 2, the amount of crystalline β-pleated sheets greatly increased by the press treatment.

TABLE 1

| Sample | Crystallinity (%) |
|---|---|
| Tussah | 47.41→49.76^{a} |
| Mori | 28.79→44.86^{a} |
| Eri | 46.89→49.29^{a} |
| Muga | 37.82→44.10^{a} |
| Thai | 9.73→40.20^{a} |
| Zein | 13.45→35.10^{a} |

TABLE 2

| Sample | Crystallinity (%) |
|---|---|
| Mori | 28.79→49.37^{a} |
| Eri | 46.89→51.80^{a} |
| Muga | 37.82→47.89^{a} |

More specifically, Tables 1 and 2 show the amounts of crystalline β-pleated sheets (crystallinity) before and after the obtained film-shaped thermoconductive material formed of protein was press-treated under 1.7 ton/cm² by the above-mentioned press machine (Model 4350.L, manufactured by Carver, Inc.). In Tables 1 and 2, the values on the left side of arrows are those measured before the press treatment and the values (suffixed with uppercase “a”) on the right side of arrows are those measured after the press treatment. The unit for crystallinity is % by weight, and the margin of error is ±2% by weight. As to the calculation of the amount of crystalline β-pleated sheets by deconvolution of the FTIR spectra, the method described in Hu et al. Macromolecules, 2006, 39, pp 6161-6170 was employed.

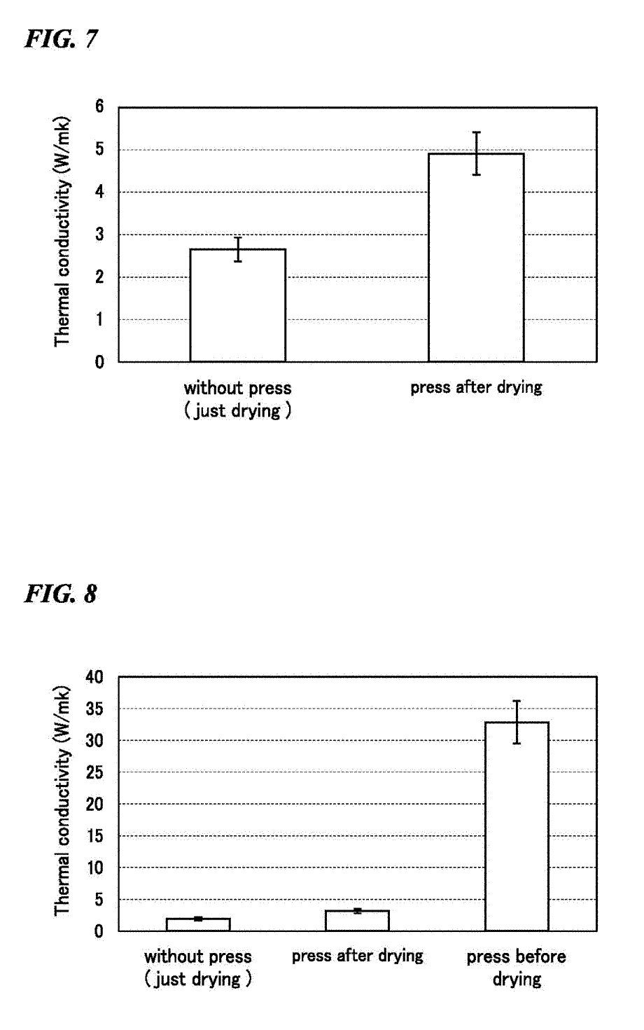

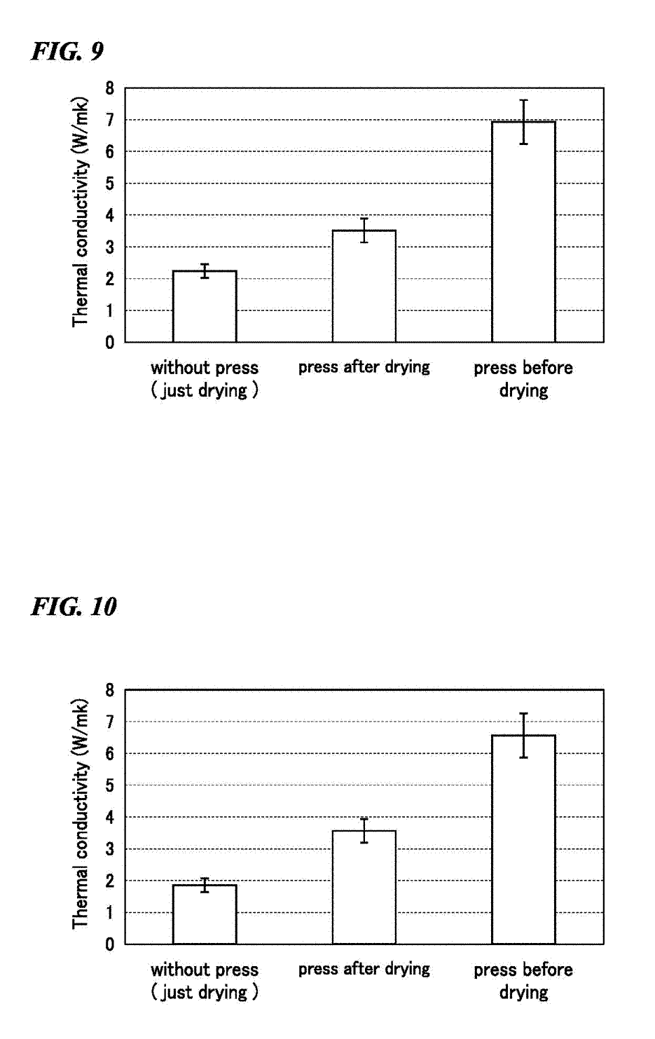

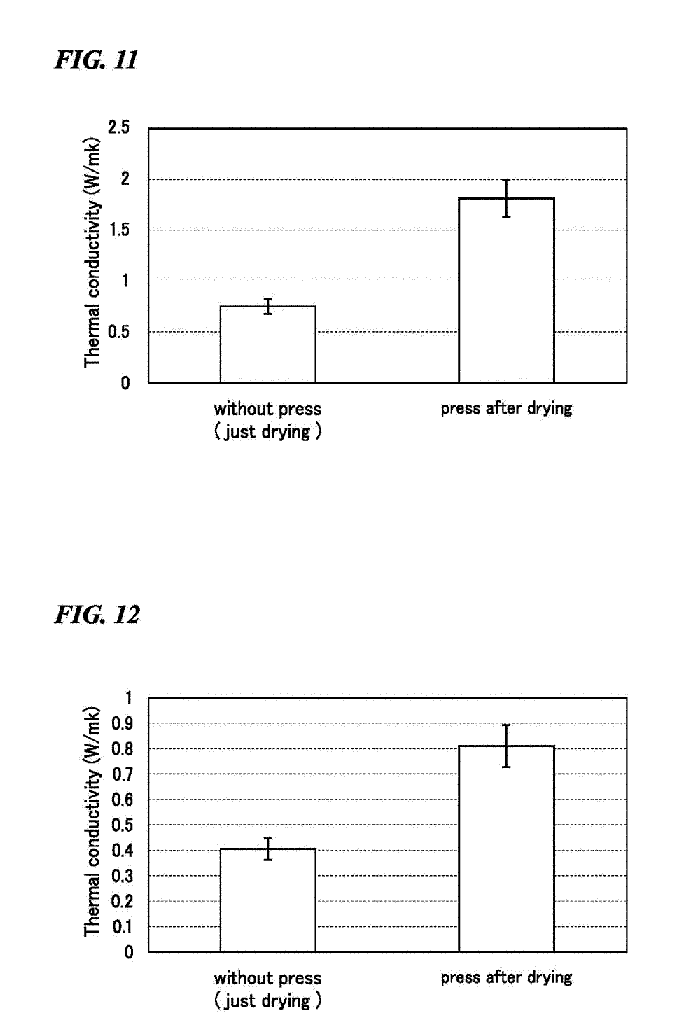

The films without the press treatment exhibited thermal conductivity in the range of 0.41 to 2.67 W/(m·K); however, the thermal conductivity of the films increased after the press treatment (see Tables 3 to 8 and FIGS. 7 to 12). From these results, it was found that the press treatment is extremely important for the purpose of improving the thermal conductivity of the thermoconductive material of the present invention.

TABLE 3
<Tussah>
Sample dimension (mm)

| | Without press | |
|---|---|---|
| | Treatment | Press-treated after |
| | (just drying) | drying |
| Length | 12.77 | 16.37 |
| Width | 3.84 | 3.38 |
| Thickness | 0.33 | 0.20 |
| Thermal | 2.67 | 4.94 |
| Conductivity | | |
| (W/(m · K)) | | |

TABLE 4
<Mori>
Sample dimension (mm)

| | Without Press | | |
|---|---|---|---|
| | Treatment | Press-treated | Press-treated Before |
| | (just drying) | After Drying | Drying |
| Lengh | 15.50 | 13.33 | 12.00 |
| Width | 1.75 | 3.03 | 1.67 |
| Thickness | 0.46 | 0.38 | 0.08 |
| Thermal | 1.91 | 3.13 | 32.99 |
| Conductivity | | | |
| (W/(m · K)) | | | |

TABLE 5
<Eri>
Sample dimension (mm)

| | Without Press | | |
|---|---|---|---|
| | Treatment | Press-treated After | Press-treated |
| | (just drying) | Drying | Before Drying |
| Length | 12.52 | 12.88 | 13.6 |
| Width | 2.17 | 1.92 | 2.216 |
| Thickness | 0.34 | 0.24 | 0.18 |
| Thermal | 2.24 | 3.52 | 6.92 |
| Conductivity | | | |
| (W/(m · K)) | | | |

TABLE 6
<Muga>
Sample dimension (mm)

| | Without Press | | |
|---|---|---|---|
| | Treatment | Press-treated After | Press-treated Before |
| | (just drying) | Drying | Drying |
| Length | 13.44 | 13.59 | 11.50 |
| Width | 2.23 | 2.69 | 2.33 |
| Thickness | 0.41 | 0.22 | 0.16 |
| Thermal | 1.85 | 3.57 | 6.57 |
| Conductivity | | | |
| (W/(m · K)) | | | |

TABLE 7
<Thai>
Sample dimension (mm)

| | Without Press | |
|---|---|---|
| | Treatment | Press-treated After |
| | (just drying) | Drying |
| Length | 21.47 | 22.76 |
| Width | 2.66 | 3.12 |
| Thickness | 0.38 | 0.23 |
| Thermal | 0.75 | 1.82 |
| Conductivity | | |
| (W/(m · K)) | | |

TABLE 8
<Zein>
Sample dimension (mm)

| | Without Press | |
|---|---|---|
| | Treatment | Press-treated After |
| | (just drying) | Drying |
| Length | 17.24 | 19.03 |
| Width | 3.50 | 4.15 |
| Thickness | 0.44 | 0.35 |
| Thermal | 0.41 | 0.81 |
| Conductivity | | |
| (W/(m · K)) | | |

FIGS. 7 to 12 show the thermal conductivity of the films without press treatment and the films after the press treatment (performed before or after drying) obtained in the Example. (Here, the thermal conductivity measured was thermal conduction of the film in a direction parallel to its surfaces, i.e., in a direction orthogonal to the thicknesswise direction of the film. Further, the “films without press treatment” mean the samples obtained by drying the films which were simply immersed in water to remove the residual calcium chloride and formic acid.) Tables 3 to 8 show the dimensions of sample films. The measurement was carried out under conditions wherein the temperature was in the range of 300 to 306 K, and the degree of vacuum was 9.0×10⁻⁵ Torr. The measurement accuracy was ±10%.

From the results shown in FIGS. 7 to 12, it can be seen that each of the films exhibited excellent thermal conductivity, which is far superior to those of synthetic resins. The films after the press treatment were completely insoluble in water. It is possible that the thermal conductivity of the films obtained in the Examples can be improved further by the addition of other thermoconductive compounds as additives.

In FIGS. 7 to 12, the results of measurements of the thermal conductivity of the films before the immersion in water are not shown. This is because the films before the immersion in water contained calcium chloride which may affect the results of measurements of the thermal conductivity.

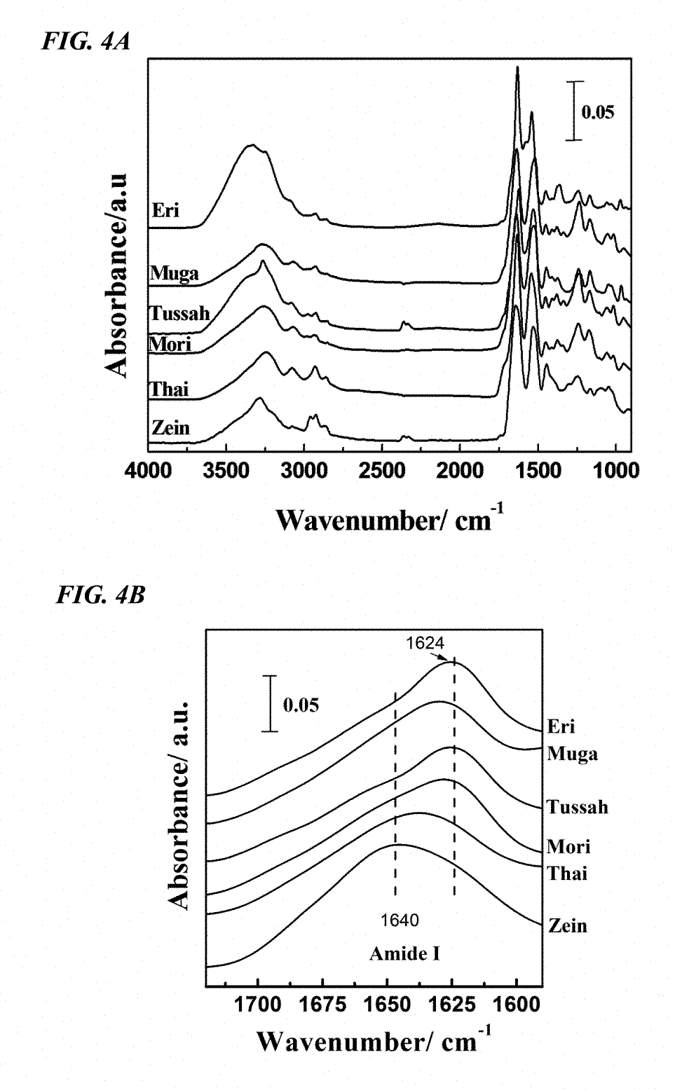

With respect to each of the films produced in the Example using the 6 types of raw material proteins, the amount of crystalline β-pleated sheets after the press treatment was measured by the FTIR analysis. FIG. 4 shows FTIR absorption spectra of the films (press-treated after drying) as measured at wavelengths of 900 to 4000 cm⁻¹. From the results shown in FIG. 4, it can be seen that the amount of crystalline β-pleated sheets increased by the press treatment with respect to all of the films produced in the Example. The results are shown in Tables 1 and 2. For example, in the case of Mori silk (Table 1), the amount of crystalline β-pleated sheets increased from 28.79% as measured before the press treatment to as high as 44.86% as measured after the press treatment. These results indicate that the amount of crystalline β-pleated sheets was increased by the press treatment.

The thermal conductivity of a solid is generally defined by the following basic formula (I):

$$k = \frac{Q * Δ x}{A * Δ T} \qquad ( I )$$

wherein k is the thermal conductivity of a solid, Δx is the thickness of a specimen (i.e., travel distance of the thermal energy), Q is the rate of heat radiated from the specimen for a predetermined period of time, A is the total area of the specimen, and ΔT is the difference in temperature (unit: Kelvin) between the initial point and final point of the measurement.

The S.I. unit for the obtained thermal conductivity values is W/m−K (Watts/meter per Kelvin).

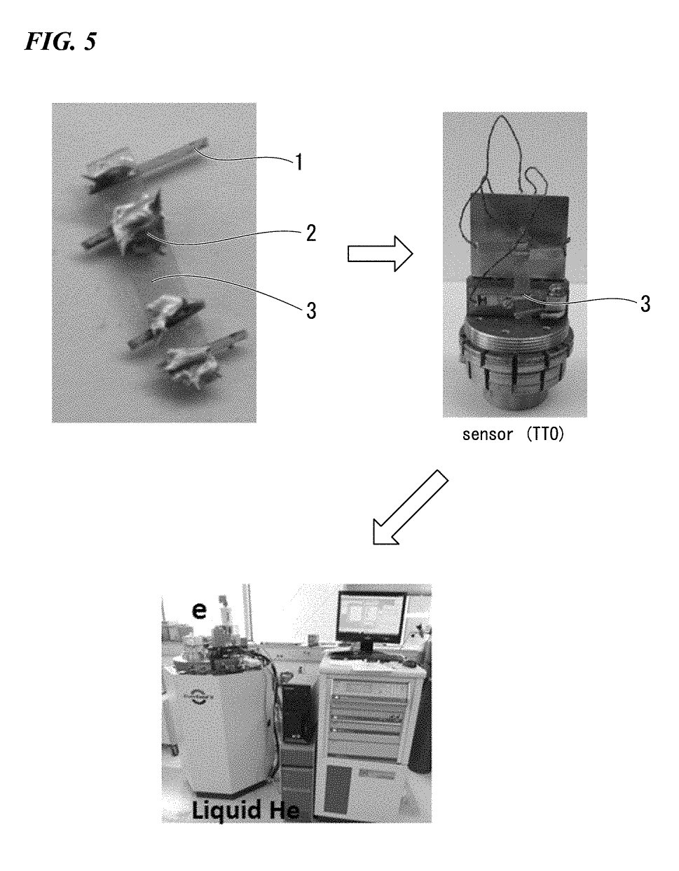

The constructions of an apparatus and a sensor for measuring the thermal conductivity of the films are respectively shown in FIG. 5 for illustrative purpose. The results of the thermal conductivity measurements performed using a system with such constructions are shown in FIGS. 7 to 12. Here, the thermal conductivity measured was thermal conduction of the film in a direction parallel to its surfaces, i.e., in a direction orthogonal to the thicknesswise direction of the film.

[Thermal Conductivity Measurements (in-Plane Direction of the Film)]

In the measurements, the Physical Property Measurement System (PPMS®) (manufactured and sold by Quantum Design, Inc.) configured with the thermal transport option (TTO) was used to evaluate the thermal conductivity of each of the films. First, the sample film was cut into a piece as described in Tables 3 to 8. Then, four copper leads were selected, each copper lead being gold plated (Part Number 4084-610, manufactured and sold by Quantum Design, Inc.).

An epoxy-based conductive adhesive containing silver was applied to one surface of each of the copper leads, and the resultant copper leads were bonded to the sample film via the conductive adhesive as shown in FIG. 5. Here, care was taken to prevent the contact between the conductive adhesives on different copper leads, i.e., to prevent the thermal conduction between the copper leads via the conductive adhesive.

Then, the sample film with the copper leads bonded thereto via the epoxy-based conductive adhesive containing silver was baked in an oven at 50 to 60° C. for 12 hours.

Then, the epoxy-based conductive adhesive containing silver was applied to other surface of each of the copper leads. Subsequently, the sample film was further baked in an oven at 50 to 60° C. for another 12 hours.

This baking was performed for two purposes, the first one being to cure the epoxy-based conductive adhesive containing silver, and the second one being to remove moisture which may affect the measured values of the thermal conductivity.

After the baking, the sample film was attached to the sensor included in the thermal conductivity accessory TTO. The copper leads were connected to the heater, hot thermometer, cold thermometer and coldfoot respectively, following instructions of the instrument manual (see FIG. 5).

The assembled thermal conductivity accessory TTO with the sample film was inserted into the PPMS tank. The tank was filled with helium gas and the gas pressure inside the tank was controlled within a range of from normal atmospheric pressure to as low as 9.0×10⁻⁵ Torr.

By the use of TTO, the thermal conductivity can be measured sequentially. The mechanism of thermal conductivity measurement operated by the PPMS software was as follows.

One end of the sample film was held at a constant temperature by maintaining good thermal contact with a low-temperature part of the sample puck of TTO. Then, the other end of the film was heated, so that a temperature gradient was established on the sample film.

### [Thermal Conductivity Measurements (Thicknesswise Direction of the Film)]

The measurements were performed with respect to the Mori silk film produced above. First, from the volume and weight of the film, the density thereof was calculated to be 1.18×10³ kg/m³. Then, the specific heat capacity of the film was measured using Differential Scanning Calorimeter (DSC) Q100 manufactured by TA Instruments. As result, the specific heat capacity of the film at 25 to 27° C. was found to be 2.52×10³ J/kgK. Thereafter, the thermal diffusivity of the film was measured by Thermowave Analyzer TA manufactured by Bethel Co., Ltd., and found to be 0.26×10⁻⁶ m²/s.

Based on these results, the thermal conductivity of the film was calculated by the following equation:

λ=αρc

wherein λ is the thermal conductivity (W/(m·K)), α is the thermal diffusivity (m²/s), ρ is the mass density (kg/m³), and c is the specific heat capacity (J/kgK).

As a result, the thermal conductivity of the film was found to be 0.77 W/(m·K). Thus, the Mori silk film had a thermal conductivity superior to that (up to approximately 0.6 W/(m·K)) of conventional polymer materials. This result confirms that a protein material treated for increasing the amount of the β-pleated sheets can exhibit high thermal conductivity in a thicknesswise direction as well.

[symbol]

Use of Thermoconductive Material

[symbol]

The thermoconductive material of the first embodiment of the present invention can be suitably used as a heat sink or a heat spreader. For example, by inserting the thermoconductive material between a heat generator and a heat dissipater, the efficiency of heat dissipation from the heat dissipater can be increased. For example, when the heat generator is a CPU, it is possible to employ a construction in which the thermoconductive material as a heat spreader is tightly attached to the surface of the CPU, and an aluminum heat sink is installed on the thermoconductive material. In such application fields as mentioned above where the thermoconductive material is installed in association with an electronic circuit, the thermoconductive material of the present embodiment is advantageous when provided in the form of a film due to the readiness of installation.

Further, the thermoconductive material of the present embodiment may be formed into a shape of a block or a column as in the case of conventional heat sinks, and used as a heat dissipater.

As mentioned above, the thermal conductivity of the thermoconductive material of the present embodiment tends to increase by the β sheet increasing treatment whereby the amount of crystalline β-pleated sheets can be increased. A preferred example of this treatment is the press treatment as also mentioned above. The press treatment may be performed either during the production in advance or during the actual use (installation).

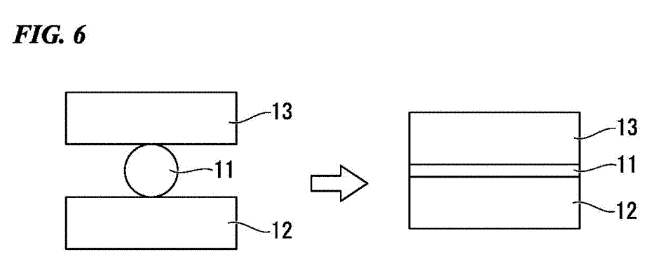

As an example of method for performing the press treatment during the use of the thermoconductive material, there can be mentioned a method in which, as shown in FIG. 6, the thermoconductive material 11 of the present embodiment which has a shape of particles, granules or pellets is sandwiched between the heat generator 12 and the heat dissipater 13, and the heat generator 12 and the heat dissipater 13 are pressed against each other so as to compress the thermoconductive material 11. Thus, the process of increasing β sheet content can be carried out by the press treatment, and the process may also be carried out by an extension and/or a shear treatment. By such a treatment, not only can the thermoconductive material 11 be adhered to the installation surfaces of the heat generator 12 and the heat dissipater 13, but the amount of crystalline β-pleated sheets in the thermoconductive material 11 can also be increased, thereby enhancing the thermal conductivity of the thermoconductive material 11. In this instance, the thermoconductive material 11 may be provided in a form of a paste obtained by mixing the thermoconductive material 11 with an appropriate binder, which paste is applied on the installation surfaces to effect the alignment of the thermoconductive material 11 contained in the paste.

The elements, combinations thereof, etc. that are explained above in connection with the specific embodiments of the present invention are mere examples, and various alterations such as addition, omission and substitution of any components, etc. may be made as long as such alterations do not deviate from the gist of the present invention. The present invention should not be limited by the above explanations and is limited only by the annexed claims.

### DESCRIPTION OF THE REFERENCE SIGNS

1 Copper lead
2 Epoxy-based conductive adhesive containing silver
3 Sample film
11 Thermoconductive material
12 Heat generator
13 Heat dissipater

1. A thermoconductive material comprising a protein and having a thermal conductivity of more than 0.6 W/(m·K).

2. The thermoconductive material according to claim 1, wherein at least a part of the protein forms crystalline β-pleated sheets, the crystalline β-pleated sheets being present in an amount of 10% by weight or more, based on the total weight of the protein.

3-4. (canceled)

5. The thermoconductive material according to claim 1, wherein the protein is fibroin or corn zein.

6. The thermoconductive material according to claim 1, which has a shape of a fiber, a film, a block, a column, a spheroid, a sphere, particles, granules or pellets.

7. (canceled)

8. A heat sink comprising the thermoconductive material of claim 6.

9. A heat spreader comprising the thermoconductive material of claim 6.

10. A method for producing a heat spreader, comprising pressing the thermoconductive material of claim 6 against an installation surface of a base to obtain a heat spreader comprising the thermoconductive material installed on the installation surface of the base.

11. A method for producing a thermoconductive material comprising a protein at least a part of which forms crystalline β-pleated sheets,
the crystalline β-pleated sheets being present in an amount of 10% by weight or more, based on the total weight of the protein, and
the thermoconductive material having a thermal conductivity of more than 0.6 W/(m·K),
the method comprising dissolving a raw material protein in a formic acid solution containing calcium chloride to obtain a protein solution, and evaporating the formic acid from the protein solution.

12. The method according to claim 11, which further comprises immersing the thermoconductive material in a polar solvent to cause at least a part of at least one of the calcium chloride or the formic acid to elute from the thermoconductive material into the polar solvent.

13. The method according to claim 12, which further comprises pressing the thermoconductive material after causing at least a part of at least one of the calcium chloride or the formic acid to elute from the thermoconductive material into the polar solvent.

14. A heat sink comprising the thermoconductive material of claim 1.

15. A heat spreader comprising the thermoconductive material of claim 1.

16. A method for producing a heat spreader, comprising pressing the thermoconductive material of claim 1 against an installation surface of a base to obtain a heat spreader comprising the thermoconductive material installed on the installation surface of the base.

17. The thermoconductive material according to claim 2, wherein the protein is fibroin or corn zein.

18. The thermoconductive material according to claim 2, which has a shape of a fiber, a film, a block, a column, a spheroid, a sphere, particles, granules or pellets.

19. A heat sink comprising the thermoconductive material of claim 2.

20. A heat sink comprising the thermoconductive material of claim 15.

21. A heat spreader comprising the thermoconductive material of claim 2.

22. A heat spreader comprising the thermoconductive material of claim 18.

23. A method for producing a heat spreader, comprising pressing the thermoconductive material of claim 2 against an installation surface of a base to obtain a heat spreader comprising the thermoconductive material installed on the installation surface of the base.

24. A method for producing a heat spreader, comprising pressing the thermoconductive material of claim 18 against an installation surface of a base to obtain a heat spreader comprising the thermoconductive material installed on the installation surface of the base.
